(19) United States
(12) Patent Application Publication
(10) Pub. No.: US 2024/0239734 A1
(43) Pub. Date: Jul. 18, 2024

(54) HYDROGENATION OF NITROBENZOIC ACID AND NITROBENZAMIDE

(71) Applicants: FMC Corporation, Philadelphia, PA (US); FMC Agro Singapore Pte. Ltd., Singapore (SG)

(72) Inventors: Ivan Sergeyevich BALDYCHEV, Wilmington, DE (US); Richard M. CORBETT, West Chester, PA (US); Rafael SHAPIRO, Wilmington, DE (US); Christina S. STAUFFER, Landenberg, PA (US)

(21) Appl. No.: 18/288,668

(22) PCT Filed: Apr. 28, 2022
(86) PCT No.: PCT/US2022/026686

§ 371 Date: Oct. 27, 2023

Related U.S. Application Data
(60) Provisional application No. 63/182,091, filed on Apr. 30, 2021.

(51) Int. Cl. C07C 51/41

(52) U.S. Cl. CPC C07C 51/418; C07C 2525/02

(57) ABSTRACT

Described herein are novel reduction reactions. Compounds prepared by the methods disclosed herein are useful for preparation of certain anthranilamide compounds that are of interest as insecticides, such as, for example, the insecticides chlorantraniliprole and cyantraniliprole.

### CROSS-REFERENCE TO RELATED APPLICATION

This application claims the benefit of U.S. Provisional Application No. 63/182,091 filed Apr. 30, 2021.

### FIELD OF INVENTION

This disclosure is directed to novel reduction reactions. Compounds prepared by the methods disclosed herein are useful for preparation of certain anthranilamide compounds that are of interest as insecticides, such as, for example, the insecticides chlorantraniliprole and cyantraniliprole.

### BACKGROUND

Conventional processes for the reduction of 3-methyl-2-nitrobenzoic acid and 2-nitro-N,3-dimethylbenzamide are subject to several industrial concerns, such as high cost, limitations of recycling, and complication operations.

The present disclosure provides novel methods useful for the reduction of 3-methyl-2-nitrobenzoic acid and 2-nitro-N,3-dimethylbenzamide. The benefits of the methods of the present disclosure compared to previous methods are numerous and include reduced cost, relatively short method steps, and simplified operation complexity.

### BRIEF DESCRIPTION

In one aspect, provided herein is a method of preparing a compound of Formula III, wherein

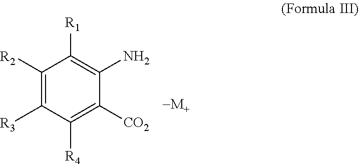

each of R₁-R₄ is independently selected from hydrogen and C₁-C₅ alkyl; and
M is a metal ion selected from sodium, potassium, calcium, and barium, the method comprising: reacting a mixture comprising
   A) a compound of Formula II, wherein

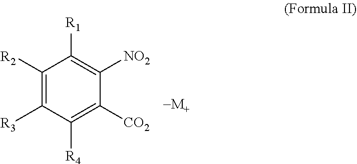

each of R₁-R₄ is independently selected from hydrogen and C₁-C₅ alkyl; and
M is a metal ion selected from sodium, potassium, calcium, and barium;
B) a reducing agent;
C) a catalyst; and
D) an aqueous solution.

In one aspect, provided herein is a method of preparing a compound of Formula V, wherein

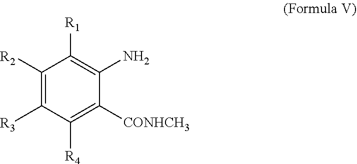

each of R₁-R₄ is independently selected from hydrogen and C₁-C₅ alkyl, the method comprising: reacting a mixture comprising
   A) a compound of Formula IV, wherein

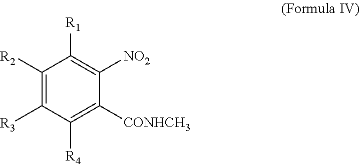

each of R₁-R₄ is independently selected from hydrogen and C₁-C₅ alkyl;
B) a reducing agent;
C) a catalyst; and
D) an organic solvent.

### DETAILED DESCRIPTION OF THE DISCLOSURE

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process or method that comprises a list of elements is not necessarily limited to only those elements but may include other elements not expressly listed or inherent to such composition, mixture, process or method.

The transitional phrase “consisting of” excludes any element, step, or ingredient not specified. If in the claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole.

The transitional phrase “consisting essentially of” is used to define a composition or method that includes materials, steps, features, components, or elements, in addition to those literally disclosed, provided that these additional materials, steps, features, components, or elements do not materially affect the basic and novel characteristic(s) of the claimed invention. The term “consisting essentially of” occupies a middle ground between “comprising” and “consisting of”.

Where an invention or a portion thereof is defined with an open-ended term such as “comprising,” it should be readily understood that (unless otherwise stated) the description should be interpreted to also describe such an invention using the terms “consisting essentially of” or “consisting of.”

Further, unless expressly stated to the contrary, “or” refers to an inclusive or and not to an exclusive or. For example, a condition A or B is satisfied by any one of the following: A is true (or present) and B is false (or not present), A is false (or not present) and B is true (or present), and both A and B are true (or present).

Also, the indefinite articles “a” and “an” preceding an element or component of the invention are intended to be nonrestrictive regarding the number of instances (i.e. occurrences) of the element or component. Therefore “a” or “an” should be read to include one or at least one, and the singular word form of the element or component also includes the plural unless the number is obviously meant to be singular.

As used herein, the term “about” means plus or minus 10% of the value.

The term “alkyl” includes, without limitation, a functional group comprising straight-chain or branched alkyl. In some aspects, the alkyl may be methyl, ethyl, n-propyl, i-propyl, or the different butyl or pentyl isomers.

The term “C₁-C₅ alkyl” includes, without limitation, a functional group comprising straight-chain or branched alkyl having one, two, three, four, or five carbon atoms.

Certain compounds of this invention can exist as one or more stereoisomers. The various stereoisomers include enantiomers, diastereomers, atropisomers and geometric isomers. One skilled in the art will appreciate that one stereoisomer may be more active and/or may exhibit beneficial effects when enriched relative to the other stereoisomer(s) or when separated from the other stereoisomer(s). Additionally, the skilled artisan knows how to separate, enrich, and/or to selectively prepare said stereoisomers.

The embodiments of this disclosure include:

Embodiment 1. A method of preparing a compound of Formula III, wherein

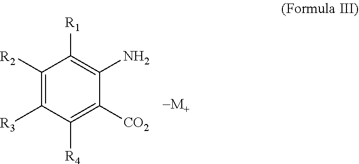

each of R₁-R₄ is independently selected from hydrogen and C₁-C₅ alkyl; and
M is a metal ion selected from sodium, potassium, calcium, and barium, the method comprising: reacting a mixture comprising
   A) a compound of Formula II, wherein

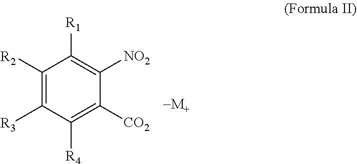

each of R₁-R₄ is independently selected from hydrogen and C₁-C₅ alkyl; and
M is a metal ion selected from sodium, potassium, calcium, and barium;
B) a reducing agent;
C) a catalyst; and
D) an aqueous solution.

Embodiment 2. The method of embodiment 1, wherein the reducing agent is hydrogen gas (H₂).

Embodiment 3. The method of embodiment 1, wherein the catalyst is in a form selected from a slurry, a pellet, a solid, a fine-grained solid, and combinations thereof.

Embodiment 4. The method of embodiment 1, wherein the catalyst is selected from nickel, Raney nickel, palladium, platinum, rhodium, gold, ruthenium, iridium, osmium, rhenium, silver, indium, germanium, beryllium, gallium, tellurium, bismuth, mercury, titanium, zirconium, hafnium, vanadium, niobium, tantalum, chromium, molybdenum, tungsten, manganese, technetium, iron, cobalt, copper, zinc, cadmium, and combinations thereof.

Embodiment 5. The method of embodiment 1, wherein the catalyst is selected from nickel, Raney nickel, palladium, platinum, palladium on carbon, and combinations thereof.

Embodiment 6. The method of embodiment 1, wherein the catalyst is dispersed on a support selected from metal oxides, zeolites, alumina, silicon carbide, carbons, and combinations thereof.

Embodiment 7. The method of embodiment 1, wherein the metal oxides are selected from Al₂O₃, SiO₂, TiO₂, and combinations thereof.

Embodiment 8. The method of embodiment 1, wherein the aqueous solution is selected from deionized water, tap water, and combinations thereof.

Embodiment 9. The method of embodiment 1, wherein the aqueous solution comprises a metal hydroxide.

Embodiment 10. The method of embodiment 1, wherein the aqueous solution is free of compounds selected from organic solvents, organic hydroxides, alkyl hydroxides, methanol, ethanol, isopropanol, and combinations thereof.

Embodiment 11. The method of embodiment 1, wherein the method step of reacting occurs at a temperature in the range of from about 80° C. to about 120° C.

Embodiment 12. The method of embodiment 1, wherein the method step of reacting occurs at a pressure in the range of from about 100 psi to about 400 psi.

Embodiment 13. The method of embodiment 1, wherein the compound of Formula III is

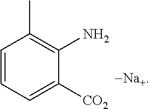

Embodiment 14. The method of embodiment 1, wherein the compound of Formula II is

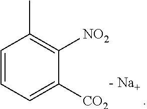

Embodiment 15. The method of embodiment 1, wherein the compound of Formula II is prepared according to a method comprising: dissolving a compound of Formula I, wherein

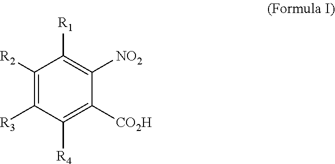

each of R₁-R₄ is independently selected from hydrogen and C₁-C₅ alkyl, in a mixture comprising
   A) the compound of Formula I;
   B) an aqueous solution; and
   C) a metal hydroxide.

Embodiment 16. The method of embodiment 15, wherein the aqueous solution is selected from deionized water, tap water, and combinations thereof.

Embodiment 17. The method of embodiment 15, wherein the aqueous solution is free of compounds selected from organic solvents, organic hydroxides, alkyl hydroxides, methanol, ethanol, isopropanol, and combinations thereof.

Embodiment 18. The method of embodiment 15, wherein the metal hydroxide is sodium hydroxide.

Embodiment 19. The method of embodiment 15, wherein the compound of Formula II is

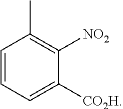

Embodiment 20. A method of preparing a compound of Formula V, wherein

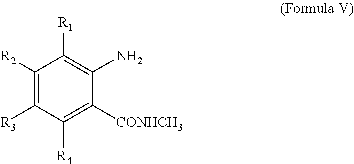

each of R₁-R₄ is independently selected from hydrogen and C₁-C₅ alkyl, the method comprising: reacting a mixture comprising
   A) a compound of Formula IV, wherein

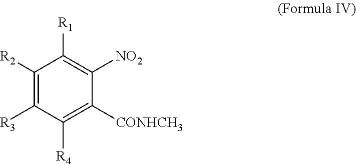

each of R₁-R₄ is independently selected from hydrogen and C₁-C₅ alkyl;
B) a reducing agent;
C) a catalyst; and
D) optionally an organic solvent.

Embodiment 21. The method of embodiment 20, wherein the reducing agent is hydrogen gas (H₂).

Embodiment 22. The method of embodiment 20, wherein the catalyst is in a form selected from a slurry, a pellet, a solid, a fine-grained solid, and combinations thereof.

Embodiment 23. The method of embodiment 20, wherein the catalyst is selected from nickel, Raney nickel, palladium, platinum, rhodium, gold, ruthenium, iridium, osmium, rhenium, silver, indium, germanium, beryllium, gallium, tellurium, bismuth, mercury, titanium, zirconium, hafnium, vanadium, niobium, tantalum, chromium, molybdenum, tungsten, manganese, technetium, iron, cobalt, copper, zinc, cadmium, and combinations thereof.

Embodiment 24. The method of embodiment 20, wherein the catalyst is selected from nickel, Raney nickel, palladium, platinum, palladium on carbon, and combinations thereof.

Embodiment 25. The method of embodiment 20, wherein the catalyst is dispersed on a support selected from metal oxides, zeolites, alumina, silicon carbide, carbons, and combinations thereof.

Embodiment 26. The method of embodiment 20, wherein the organic solvent is selected from methanol, ethanol, isopropanol, and combinations thereof.

Embodiment 27. The method of embodiment 20, wherein the compound of Formula V is

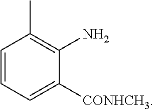

Embodiment 28. The method of embodiment 20, wherein the compound of Formula IV is

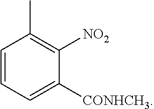

In one aspect, a compound of Formula III is prepared according to a method represented by Scheme 1. The R groups and M group are as defined anywhere in this disclosure.

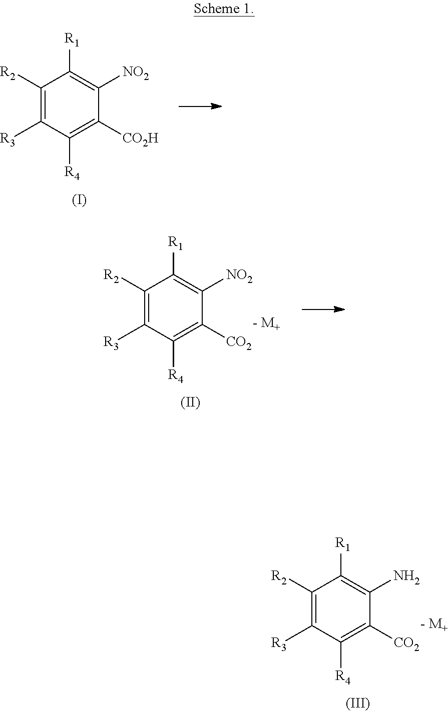

In one aspect, a compound of Formula V is prepared according to a method represented by Scheme 2. The R groups and M group are as defined anywhere in this disclosure.

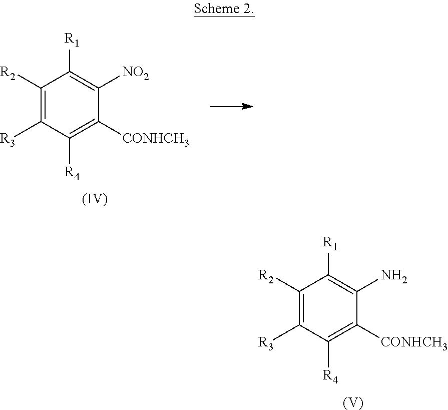

In one aspect, sodium 3-methyl-2-aminobenzoate is prepared according to a method represented by Scheme 3.

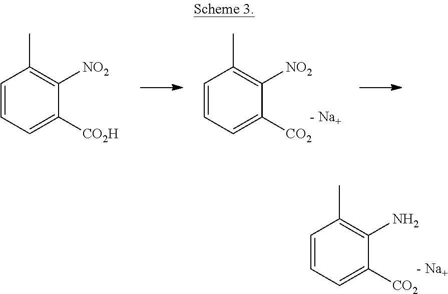

In one aspect, 2-amino-N,3-dimethylbenzamide is prepared according to a method represented by Scheme 4.

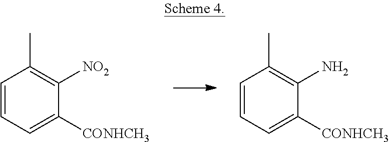

In one aspect, a compound of Formula II is prepared according to a method represented by Scheme 5. The R groups and M group are as defined anywhere in this disclosure.

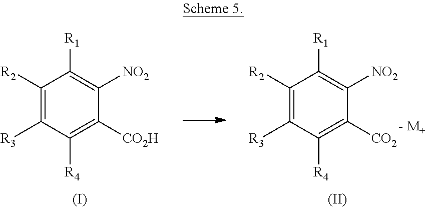

This aspect includes dissolving a compound of Formula I in an aqueous solution in the presence of a metal hydroxide. In some embodiments, the aqueous solution is selected from deionized water, tap water, and combinations thereof. In some embodiments, the aqueous solution does not comprise an organic solvent. In some embodiments, the aqueous solution does not comprise an organic hydroxide. In some embodiments, the aqueous solution does not comprise an alkyl hydroxide. In some embodiments, the aqueous solution does not comprise methanol or ethanol or isopropanol. In some embodiments, the aqueous solution is free of compounds selected from organic solvents, organic hydroxides, alkyl hydroxides, methanol, ethanol, isopropanol, and combinations thereof.

In some embodiments, the metal hydroxide is selected from alkali hydroxide, alkaline earth metal hydroxide, and combinations thereof. In some embodiments, the metal hydroxide is selected from sodium hydroxide, potassium hydroxide, and combinations thereof. In some embodiments, the metal hydroxide is selected from calcium hydroxide, barium hydroxide, and combinations thereof.

In some embodiments, the method step of dissolving occurs at room temperature.

In some embodiments the dissolving step occurs at 60° C. or higher.

In some embodiments, the method step of dissolving occurs at room pressure.

In one aspect, a compound of Formula III is prepared according to a method represented by Scheme 6. The R groups and M group are as defined anywhere in this disclosure.

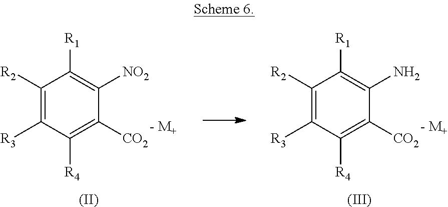

This aspect includes reacting a compound of Formula II with a reducing agent in an aqueous solution in the presence of a catalyst. In some embodiments, the reducing agent is hydrogen gas (H₂).

In some embodiments, the aqueous solution is selected from deionized water, tap water, and combinations thereof. In some embodiments, the aqueous solution does not comprise an organic solvent. In some embodiments, the aqueous solution does not comprise an organic hydroxide. In some embodiments, the aqueous solution does not comprise an alkyl hydroxide. In some embodiments, the aqueous solution does not comprise methanol or ethanol or isopropanol. In some embodiments, the aqueous solution is free of compounds selected from organic solvents, organic hydroxides, alkyl hydroxides, methanol, ethanol, isopropanol, and combinations thereof.

In some embodiments, the aqueous solution comprises a metal hydroxide. In some embodiments, the metal hydroxide is selected from alkali hydroxide, alkaline earth metal hydroxide, and combinations thereof. In some embodiments, the metal hydroxide is selected from sodium hydroxide, potassium hydroxide, and combinations thereof. In some embodiments, the metal hydroxide is selected from calcium hydroxide, barium hydroxide, and combinations thereof.

In some embodiments, the catalyst comprises a metal selected from transition metals, titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, mercury, and combinations thereof.

In some embodiments, the catalyst comprises a metal selected from nickel, Raney nickel, palladium, platinum, rhodium, gold, ruthenium, iridium, osmium, rhenium, silver, indium, germanium, beryllium, gallium, tellurium, bismuth, mercury, titanium, zirconium, hafnium, vanadium, niobium, tantalum, chromium, molybdenum, tungsten, manganese, technetium, iron, cobalt, copper, zinc, cadmium, and combinations thereof.

In some embodiments, the catalyst comprises a metal selected from nickel, aluminum, palladium, palladium/carbon (Pd/C), and combinations thereof.

In some embodiments, the catalyst is dispersed on a support selected from metal oxides, zeolites, alumina, silicon carbide, carbons, and combinations thereof. In some embodiments the metal oxides are selected from Al₂O₃, SiO₂, TiO₂, and combinations thereof.

In some embodiments, the catalyst is selected from nickel catalysts, Nickel Raney catalysts, Pd/C catalysts, and combinations thereof. In some embodiments, the catalyst is in a form selected from a slurry, a pellet, a solid, a fine-grained solid, and combinations thereof. In some embodiments, the catalyst is preferred in a form selected from a pellet, a solid, a fine-grained solid, and combinations thereof, as this form allows for easier handling and improved safety profile.

In some embodiments, the catalyst is provided directly to the aqueous solution. In some embodiments, the catalyst is provided to the aqueous solution in a catalyst holder.

In some embodiments, the method step of reacting comprises continuously providing the reducing agent to the aqueous solution. In some embodiments, the method step of reacting comprises continuously providing the reducing agent to the aqueous solution.

In some embodiments, the method step of reacting comprises discretely providing the reducing agent to the aqueous solution. In some embodiments, the method step of reacting comprises providing the reducing agent to the aqueous solution at least once. In some embodiments, the method step of reacting comprises providing the reducing agent to the aqueous solution at least twice.

In some embodiments, the method step of reacting comprises stirring the aqueous solution. In some embodiments, the method step of reacting comprises stirring the aqueous solution at a rate of at least about 50 rotations per minute (RPM), at least about 100 RPM, at least about 200 RPM, at least about 300 RPM, at least about 400 RPM, at least about 500 RPM, at least about 600 RPM, at least about 700 RPM, at least about 800 RPM, at least about 900 RPM, at least about 1000 RPM, at least about 1100 RPM, or at least about 1200 RPM.

In some embodiments, the method step of reacting occurs at a temperature in the range of from about 50° C. to about 120° C. In some embodiments, the method step of reacting occurs at a temperature in the range of from about 60° C. to about 110° C. In some embodiments, the method step of reacting occurs at a temperature in the range of from about 80° C. to about 100° C.

In some embodiments, the method step of reacting occurs at a pressure in the range of from about 30 psi to about 400 psi. In some embodiments, the method step of reacting occurs at a pressure in the range of from about 100 psi to about 400 psi. In some embodiments, the method step of reacting occurs at a pressure in the range of from about 100 psi to about 200 psi. In some embodiments, the method step of reacting occurs at a pressure in the range of from about 300 psi to about 400 psi.

In one aspect, a compound of Formula V is prepared according to a method represented by Scheme 7. The R groups are as defined anywhere in this disclosure.

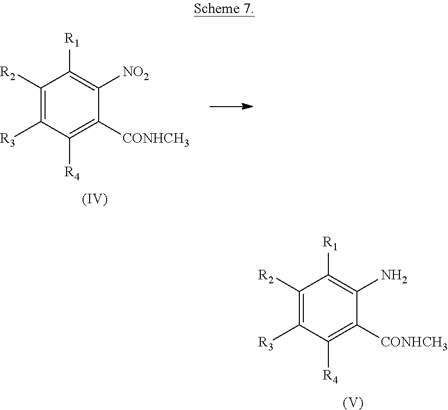

This aspect includes reacting a compound of Formula IV with a reducing agent in an organic solvent in the presence of a catalyst. In some embodiments, the reducing agent is hydrogen gas (H₂).

In some embodiments, the organic solvent comprises an organic hydroxide selected from alkyl hydroxides, methanol, ethanol, isopropanol, and combinations thereof. In some embodiments, the organic solvent is methanol or ethanol.

In some embodiments, the catalyst comprises a metal selected from transition metals, titanium, zirconium, hafnium, vanadium, niobium, tantalum, chromium, molybdenum, tungsten, manganese, technetium, rhenium, iron, ruthenium, osmium, cobalt, rhodium, iridium, nickel, palladium, platinum, copper, silver, gold, zinc, cadmium, mercury, and combinations thereof.

In some embodiments, the catalyst comprises a metal selected from nickel, Raney nickel, palladium, platinum, rhodium, gold, ruthenium, iridium, osmium, rhenium, silver, indium, germanium, beryllium, gallium, tellurium, bismuth, mercury, titanium, zirconium, hafnium, vanadium, niobium, tantalum, chromium, molybdenum, tungsten, manganese, technetium, iron, cobalt, copper, zinc, cadmium, and combinations thereof.

In some embodiments, the catalyst comprises a metal selected from nickel, aluminum, palladium, and combinations thereof.

In some embodiments, the catalyst is dispersed on a support selected from metal oxides, zeolites, alumina, silicon carbide, carbons, and combinations thereof. In some embodiments the metal oxides are selected from Al₂O₃, SiO₂, TiO₂, and combinations thereof.

In some embodiments, the catalyst is selected from nickel catalysts, Raney nickel catalysts, Pd/C catalysts, and combinations thereof. In some embodiments, the catalyst is in a form selected from a slurry, a pellet, a solid, a fine-grained solid, and combinations thereof.

In some embodiments, the catalyst is provided directly to the organic solvent. In some embodiments, the catalyst is provided to the organic solvent in a catalyst holder.

In some embodiments, the method step of reacting comprises continuously providing the reducing agent to the organic solvent. In some embodiments, the method step of reacting comprises continuously providing the reducing agent to the organic solvent.

In some embodiments, the method step of reacting comprises discretely providing the reducing agent to the organic solvent. In some embodiments, the method step of reacting comprises providing the reducing agent to the organic solvent at least once. In some embodiments, the method step of reacting comprises providing the reducing agent to the organic solvent at least twice.

In some embodiments, the method step of reacting comprises stirring the organic solvent. In some embodiments, the method step of reacting comprises stirring the organic solvent at a rate of at least about 50 rotations per minute (RPM), at least about 100 RPM, at least about 200 RPM, at least about 300 RPM, at least about 400 RPM, at least about 500 RPM, at least about 600 RPM, at least about 700 RPM, at least about 800 RPM, at least about 900 RPM, at least about 1000 RPM, at least about 1100 RPM, or at least about 1200 RPM.

In some embodiments, the method step of reacting occurs at a temperature in the range of from about 50° C. to about 120° C. In some embodiments, the method step of reacting occurs at a temperature in the range of from about 80° C. to about 100° C. In some embodiments, the method step of reacting occurs at a temperature in the range of from about 60° ° C. to about 110° C. In some embodiments, the method step of reacting occurs at a temperature in the range of from about 80° C. to about 100° C.

In some embodiments, the method step of reacting occurs at a pressure in the range of from about 30 psi to about 400 psi. In some embodiments, the method step of reacting occurs at a pressure in the range of from about 100 psi to about 400 psi. In some embodiments, the method step of reacting occurs at a pressure in the range of from about 100 psi to about 200 psi. In some embodiments, the method step of reacting occurs at a pressure in the range of from about 300 psi to about 400 psi.

### EXAMPLES

Without further elaboration, it is believed that one skilled in the art using the preceding description can utilize the present invention to its fullest extent. The following Examples are, therefore, to be construed as merely illustrative, and not limiting of the disclosure in any way whatsoever. The starting material for the following Examples may not have necessarily been prepared by a particular preparative run whose procedure is described in other Examples. It also is understood that any numerical range recited herein includes all values from the lower value to the upper value. For example, if a range is stated as 10-50, it is intended that values such as 12-30, 20-40, or 30-50, etc., are expressly enumerated in this specification. These are only examples of what is specifically intended, and all possible combinations of numerical values between and including the lowest value and the highest value enumerated are to be considered to be expressly stated in this application.

### Equipment.

The Examples demonstrated herein were obtained utilizing a 150 mL pressure reactor with overhead stirring and a gas feed system. The catalyst holder was a spinning basket holder with a pumping impeller and a wire mesh.

The high-performance liquid chromatograph (HPLC) instrument included an analytical column. Gradient separations were performed. Detection was by UV.

### Example 1. Reduction of 3-methyl-2-nitrobenzoic acid to sodium 3-methyl-2-aminobenzoate Using a Raney Nickel Catalyst

TABLE 1
Reactants.

| | Reactant | | | |
|---|---|---|---|---|
| | 3-methyl-2- | Reactant | Catalyst | |
| | nitrobenzoic | 50% aq. | 50 wt % aq. | Solvent |
| | acid | NaOH | Raney-Ni | H₂O |
| Molecular | 181 | 138.21 | 58.69 | 18 |
| weight (g/mol) | | | | |
| Mass (g) | 1.000 | 1.604 | 0.065 | 70.0 |

A 150 ml pressure reactor with overhead stirring was charged with water, Raney-Nickel catalyst, and 1.05 equivalents of 50% aqueous sodium hydroxide. About 0.065 grams of Raney-Nickel slurry (50% water) was charged, so the mass equivalence to starting material was about 3.24 wt %. The 3-methyl-2-nitrobenzoic acid was charged and produced a thin greenish-colored solution.

The reactor was sealed and pressure-purged with N₂ three times to remove air. The reactor was pressure-tested using N₂ and then the reactor was pressure-purged with hydrogen gas (H₂) three times. The reactor was pressurized with H₂ to starting pressure (300 psi) and hydrogen line was kept open, so the system was continuously supplied with H₂ as it was used up during reaction. The reactor agitation was set at 800 RPM and was heated to 80-100° C. Hydrogen gas was fed into the reactor for one hour.

After reaction was considered complete, the reactor was cooled, the pressure was vented, and a sample was taken. As shown in Table 2, HPLC analysis determined that the starting material was almost completely converted.

TABLE 2
Analytical results

| | sodium 3-methyl- | 3-methyl-2- |
|---|---|---|
| Identity | 2-aminobenzoate | nitrobenzoic acid |
| % area | 98.34% | 0.04% |

### Example 2. Reduction of 3-methyl-2-nitrobenzoic acid to sodium 3-methyl-2-aminobenzoate using a Raney Nickel Catalyst

TABLE 3
Reactants.

| | Reactant | | | |
|---|---|---|---|---|
| | 3-methyl-2- | Reactant | Catalyst | |
| | nitrobenzoic | 50% aq. | 50 wt % aq. | Solvent |
| | acid | NaOH | Raney-Ni | H₂O |
| Molecular | 181 | 138.21 | 58.69 | 18 |
| weight (g/mol) | | | | |
| Mass (g) | 5.000 | 8.018 | 0.065 | 70.0 |

A 150 ml pressure reactor with overhead stirring was charged with water, Raney-Nickel catalyst, and 50% aqueous sodium hydroxide. About 0.065 grams of Raney-Nickel slurry (50 wt % water) was charged. Then NaOH and 3-methyl-2-nitrobenzoic acid were charged and produced a thin, greenish-colored solution.

The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (300 psi) and hydrogen line was kept open so the system was continuously supplied with H₂ as it was used up during reaction.

The reactor agitation was set to 800 RPM. It was heated to 100° C. Hydrogen gas was fed into the reactor for one hour.

After a period of time, the reactor was cooled, the pressure was vented, and a sample was taken. HPLC analysis results are shown in Table 4.

TABLE 4
Analytical results

| | sodium 3-methyl- | 3-methyl-2- |
|---|---|---|
| Identity | 2-aminobenzoate | nitrobenzoic acid |
| % area | 78.38% | 21.62% |

### Example 3. Reduction of 3-methyl-2-nitrobenzoic acid to sodium 3-methyl-2-aminobenzoate Using a Raney Nickel Catalyst

TABLE 6
Reactants.

| | Reactant | | | |
|---|---|---|---|---|
| | 3-methyl-2- | Reactant | Catalyst | |
| | nitrobenzoic | 50% aq. | 50% aq. | Solvent |
| | acid | NaOH | Raney-Ni | H₂O |
| Molecular | 181 | 138.21 | 58.69 | 18 |
| weight (g/mol) | | | | |
| Mass (g) | 10.00 | 16.035 | 0.100 | 70.0 |

A 150 ml pressure reactor with overhead stirring was charged with water, Raney-Nickel catalyst, and 50% aqueous sodium hydroxide. NaOH and 3-methyl-2-nitrobenzoic acid were charged and produced a thin, greenish-colored solution.

The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (300 psi) and hydrogen line was kept open so the system was continuously supplied with H₂ as it was used up during reaction.

The reactor agitation was set to 600 RPM. The reactor was heated to 100° C. Hydrogen gas was fed into the reactor for one hour.

After a period of time, the reactor was cooled, the pressure was vented, and a sample was taken. HPLC analysis results are shown in Table 7.

TABLE 7
Analytical results.

| | sodium 3-methyl- | 3-methyl-2- |
|---|---|---|
| Identity | 2-aminobenzoate | nitrobenzoic acid |
| % area | 3.11% | 96.89% |

The reaction was then continued. The reactor was stirred to 800 RPM. It was pressurized with H₂ to 300 psi(a) and then closed off from H₂ supply. The reactor was then heated to 100° ° C.

After a period of time, the reactor was cooled, the pressure was vented, and the reaction mass was removed from the reactor. A sample was taken. HPLC analysis is shown in Table 8.

TABLE 8
Analytical results.

| | sodium 3-methyl-2- | 3-methyl-2- |
|---|---|---|
| | aminobenzoate | nitrobenzoic acid |
| % area | 9.56% | 79.60% |

### Example 4. Reduction of 3-methyl-2-nitrobenzoic acid to sodium 3-methyl-2-aminobenzoate Using a Pd/C Slurry Catalyst

TABLE 9. Reactants.

| | Reactant | | Catalyst | |
|---|---|---|---|---|
| | 97% 3-methyl- | Reactant | 5% Pd/C | |
| | 2-nitrobenzoic | 30% aq. | slurry (50% | Solvent |
| | acid | NaOH | water wet) | H₂O |
| Molecular | 181 | 138.21 | 106.42 | 18 |
| weight (g/mol) | | | | |
| Mass (g) | 1257 | 943 | 32.6 | 2514 |

A 150 ml pressure reactor with overhead stirring was charged with water and 3-methyl-2-nitrobenzoic acid. 30% aqueous sodium hydroxide was charged and produced a thin, greenish-colored solution.

The reactor was sealed and pressure-purged with N₂ three times to remove air. The reactor was pressure-tested using N₂. The reactor was then charged with a palladium on carbon (Pd/C) slurry catalyst. It was again purged with N₂. The reactor was then pressure-purged with hydrogen gas (H₂) three times. The reactor was pressurized with H₂ to starting pressure (300 psi).

The reactor was heated to 60-80° C. Hydrogen gas was sparged into the reaction mass at such a rate as to maintain the temperature between 60-80° C. and pressure between 1.0-5.0 atmospheres. After hydrogen gas uptake ceased, the reaction mass was held for an additional 30 minutes to ensure complete conversion. Pressure was then released, and the reactor was purged with nitrogen.

The reaction mass was passed through a filter at 60-80° C. to remove catalyst. Recycling is conveniently carried out by back flushing the filter with water.

The yield of sodium 3-methyl-2-nitrobenzoate was about 98%.

### Example 5. Reduction of 3-methyl-2-nitrobenzoic acid to sodium 3-methyl-2-aminobenzoate Using a Pellet Pd/C Catalyst

TABLE 10
Reactants.

| | Reactant | | | |
|---|---|---|---|---|
| | 97% 3-methyl- | Reactant | Catalyst | |
| | 2-nitrobenzoic | 50% aq. | 2 wt % Pd/C | Solvent |
| | acid | NaOH | (3 mm pellets) | H₂O |
| Molecular | 181 | 138.21 | 106.42 | 18 |
| weight (g/mol) | | | | |
| Mass (g) | 4.9 | 8.5 | 0.62 | 80.0 |

A 150 ml pressure reactor with overhead stirring and a spinning catalyst basket holder was charged with water and 50% aqueous sodium hydroxide. NaOH and 3-methyl-2-nitrobenzoic acid were charged and produced a thin, greenish-colored solution.

The spinning catalyst basket holder was charged with a pelletized palladium on carbon (Pd/C) catalyst. The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (170 psi).

The reactor agitation was set to 1000 RPM. It was heated to 100° C. Hydrogen gas was cut off from the reactor. As pressure dropped, the reactor was re-pressurized twice with H₂. Hydrogen uptake started at about 75° C. and stopped in 80 minutes. Once pressure stopped changing and hydrogen was no longer being consumed, the reaction was deemed complete.

After the reaction was complete, the reactor was cooled, the pressure was vented, and a sample was taken. HPLC analysis results are shown in Table 11.

TABLE 11
Analytical results

| | sodium 3-methyl-2- | 3-methyl-2- |
|---|---|---|
| | aminobenzoate | nitrobenzoic acid |
| % area | 97.97% | 0.09% |

### Example 6. Reduction of 3-methyl-2-nitrobenzoic acid to sodium 3-methyl-2-aminobenzoate Using a Re-Used Pd/C Catalyst from Example 5

TABLE 12. Reactants.

| | Reactant | | | |
|---|---|---|---|---|
| | 97% 3-methyl- | Reactant | Catalyst | |
| | 2-nitrobenzoic | 50% aq. | 2 wt % Pd/C | Solvent |
| | acid | NaOH | (3 mm pellets) | H₂O |
| Molecular | 181 | 138.21 | 106.42 | 18 |
| weight (g/mol) | | | | |
| Mass (g) | 5.1 | 8.4 | 0.62 | 80.0 |

A 150 ml pressure reactor with overhead stirring and a spinning catalyst basket holder was charged with water and 50% aqueous sodium hydroxide. NaOH and 3-methyl-2-nitrobenzoic acid were charged and produced a thin, greenish-colored solution.

The spinning catalyst basket holder already contained 0.62 g of the pelletized palladium on carbon (Pd/C) catalyst from Example 5. The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (340 psi).

The reactor was stirred to 1100 RPM. It was heated to 100° C. Hydrogen gas was cut off from the reactor. As pressure dropped, the reactor was re-pressurized once with H₂. The hydrogen uptake started at about 75° C. and completed in 50 minutes. Once pressure stopped changing and hydrogen was no longer being consumed, the reaction was deemed complete.

After a period of time, the reactor was cooled, the pressure was vented, and a sample was taken. HPLC analysis results are shown in Table 13.

TABLE 13
Analytical results.

| | sodium 3-methyl-2- | 3-methyl-2- |
|---|---|---|
| | aminobenzoate | nitrobenzoic acid |
| % area | 98.34% | 0.11% |

### Example 7. Reduction of 2-nitro-N,3-dimethylbenzamide to 2-amino-N,3-dimethylbenzamide Using a Pelletized Nickel Catalyst

TABLE 14
Reactants.

| | Reactant | Catalyst | |
|---|---|---|---|
| | 2-nitro-N,3- | Supported Ni | Solvent |
| | dimethylbenzamide | catalyst | Methanol |
| Molecular | 194.18 | | 18 |
| weight (g/mol) | | | |
| Mass (g) | 5.0 | 0.050-0.25 | 20.0 |

A 150 ml pressure reactor with overhead stirring and a spinning catalyst basket holder was charged with methanol. 2-nitro-N,3-dimethylbenzamide was charged.

The spinning catalyst basket holder was charged with a supported nickel catalyst. The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (170 psi).

The reactor agitation was set to 1000 RPM. It was heated to 100° C. Hydrogen gas was cut off from the reactor. As pressure dropped, the reactor was re-pressurized with H₂. Once pressure stopped changing and hydrogen was no longer being consumed, the reaction was deemed complete.

After a period of time, the reactor was cooled, the pressure was vented, and a sample was taken. The sample was analyzed by HPLC.

The reaction mass was removed from the reactor and the catalyst basket holder was rinsed with water. The washing water was added to the reaction mass.

### Example 8. Reduction of 2-nitro-N,3-dimethylbenzamide to 2-amino-N,3-dimethylbenzamide Using Re-Used Pelletized Nickel Catalyst from Example 7

TABLE 15
Reactants.

| | Reactant | Catalyst | |
|---|---|---|---|
| | 2-nitro-N,3- | Supported Ni | Solvent |
| | dimethylbenzamide | catalyst | Methanol |
| Molecular | 194.18 | | 18 |
| weight (g/mol) | | | |
| Mass (g) | 5.0 | 0.050-0.25 | 20.0 |

A 150 ml pressure reactor with overhead stirring and a spinning catalyst basket holder was charged with methanol. 2-nitro-N,3-dimethylbenzamide was charged.

The spinning catalyst basket holder already contained the supported nickel catalyst of Example 7. The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (340 psi).

The reactor agitation was set to 1100 RPM. It was heated to 100° C. Hydrogen gas was cut off from the reactor. Once pressure stopped changing and hydrogen was no longer being consumed, the reaction was deemed complete.

After a period of time, the reactor was cooled, the pressure was vented, and a sample was taken.

### Example 9. Reduction of 2-nitro-N,3-dimethylbenzamide to 2-amino-N,3-dimethylbenzamide Using a Raney Nickel Catalyst

TABLE 16
Reactants.

| | Reactant | | | |
|---|---|---|---|---|
| | 2-nitro-N,3- | Catalyst | | |
| | dimethyl- | 50 wt % aq. | Solvent | Solvent |
| | benzamide | Raney nickel | Water | Methanol |
| Molecular | 194.18 | 58.69 | 18 | |
| weight (g/mol) | | | | |
| Mass (g) | 5.00 | 0.4660 | 50 | |
| Volume (mL) | | | | 100 |

A 250 ml round bottom flask was charged with 5 grams of 2-nitro-N,3-dimethylbenzamide and 50 grams of water, then stirred to result in a thin white slurry. Methanol (100 mL) was then added in 5 mL increments until the solids dissolved.

A 600 mL pressure reactor with overhead stirring was charged with the mixture from the round bottom flask. The reactor was charged with the Raney nickel catalyst slurry. The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (350 psi).

The reactor agitation was set to 500 RPM. It was heated to 100° C. Hydrogen gas was cut off from the reactor. As pressure dropped, the reactor was re-pressurized once with H₂. Once pressure stopped changing (i.e. hydrogen was no longer being consumed), the reaction was deemed complete.

After a period of time, the reactor was cooled and the pressure was vented.

A sample was analyzed by HPLC and results are shown in Table 17.

TABLE 17
Analytical results.

| | 2-amino-N,3- | 2-nitro-N,3- |
|---|---|---|
| | dimethylbenzamide | dimethylbenzamide |
| % area | 68.92% | 4.92% |

### Example 10. Reduction of 2-nitro-N,3-dimethylbenzamide to 2-amino-N,3-dimethylbenzamide Using a Raney Nickel Catalyst

TABLE 18
Reactants.

| | Reactant | Catalyst | |
|---|---|---|---|
| | 2-nitro-N,3- | 50wt % aq. Raney | Solvent |
| | dimethylbenzamide | nickel, slurry | Methanol |
| Molecular | 194.18 | 58.69 | |
| weight (g/mol) | | | |
| Mass (g) | 5.00 | 0.3590 | 150 |

A 250 mL round bottom flask was charged with 10 grams of 2-nitro-N,3-dimethylbenzamide and 80 grams of methanol to form a thin white slurry. Methanol (150 g) was then further added until the solids dissolved.

A 600 mL pressure reactor with overhead stirring was charged with the mixture from the round bottom flask. The reactor was also charged with the Raney nickel catalyst slurry. The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (400 psi).

The reactor agitation was set to 500 RPM. It was heated to 100° C. Hydrogen gas was cut off from the reactor. As pressure dropped, the reactor was re-pressurized once with H₂. Once pressure stopped changing (i.e. hydrogen was no longer being consumed), the reaction was deemed complete.

After the reaction was complete (about 120 minutes), the reactor was cooled and the pressure was vented.

A sample was analyzed by HPLC. The analysis results are in Table 19.

TABLE 19
Analytical results.

| | 2-amino-N,3- | 2-nitro-N,3- |
|---|---|---|
| | dimethyl- | dimethyl- |
| | benzamide | benzamide |
| % area | 84.7% | Non-detect |

### Example 11. Reduction of 2-nitro-N,3-dimethylbenzamide to 2-amino-N,3-dimethylbenzamide Using a Raney Nickel Catalyst

TABLE 20
Reactants.

| | Reactant | Catalyst | | |
|---|---|---|---|---|
| | 2-nitro-N,3- | 50 wt % aq. | Reactant | |
| | dimethyl- | Raney nickel, | Hydrogen | Solvent |
| | benzamide | slurry | (H₂) | Methanol |
| Molecular | 194.18 | 58.69 | 2 | 32.04 |
| weight (g/mol) | | | | |
| Mass (g) | 20.00 | 1.440 | 0.412 | 71.2 |

A 250 mL round bottom flask was charged with 10 grams of 2-nitro-N,3-dimethylbenzamide and 80 grams of methanol to form a thin white slurry. Methanol (90 mL) was then further added until the solids dissolved.

A 600 mL pressure reactor with overhead stirring was charged with the mixture from the round bottom flask. The reactor was also charged with the Raney nickel catalyst slurry. The reactor was sealed and pressure-purged with N₂ three times to remove air. The reactor was pressure-tested using. The reactor was then pressure-purged with hydrogen gas (H₂) three times. The reactor was pressurized with H₂ to starting pressure (150 psi).

The reactor agitation was set to about 430 RPM. It was heated to 65° C. Hydrogen gas was cut off from the reactor. As pressure dropped, the reactor was re-pressurized four times with H₂. Once pressure stopped changing (i.e. hydrogen was no longer being consumed), the reaction was deemed complete.

After the reaction was complete (around 300 minutes), the reactor was cooled and the pressure was vented.

A sample was analyzed by HPLC. The analysis results are shown in Table 21.

TABLE 21
Analytical results.

| | 2-amino-N,3- | 2-nitro-N,3- |
|---|---|---|
| | dimethylbenzamide | dimethylbenzamide |
| % area | 75.0% | 4.0% |

Weight % analysis was also performed on the reaction mass

TABLE 22
Weight % Analysis.

| | | sodium 3- |
|---|---|---|
| | 2-amino-N,3- | methyl-2- |
| | dimethylbenzamide | aminobenzoate |
| Appearance | (wt %) | (wt %) |
| Opaque black solution | 15.74 | 2.10 |

The yield of 2-amino-N,3-dimethylbenzamide based on wt % analysis was about 88%.

### Example 12. Reduction of 2-nitro-N,3-dimethylbenzamide to 2-amino-N,3-dimethylbenzamide Using a Pellet Pd/C Catalyst

TABLE 23
Reactants.

| | | Catalyst | | | |
|---|---|---|---|---|---|
| | Reactant | 2 wt % | | Product | |
| | 2-nitro-N,3- | Pd/C (3 mm | Solvent | 2-amino-N,3- | Product |
| | dimethylbenzamide | pellets) | Methanol | dimethylbenzamide | Water |
| Molecular | 194.18 | 106.42 | 32 | 151.06 | 18 |
| weight (g/mol) | | | | | |
| Mass (g) | 20.00 | 0.550 | 80.00 | 15.6 | 3.71 |

A 600 mL pressure reactor with overhead stirring and a spinning catalyst basket holder was charged with 2-nitro-N,3-dimethylbenzamide and methanol.

The spinning catalyst basket holder was charged with a pelletized palladium on carbon (Pd/C) catalyst. The reactor was sealed and pressure-purged with N₂ three times to remove air. The reactor was pressure-tested using. The reactor was then pressure-purged with hydrogen gas (H₂) three times. The reactor was pressurized with H₂ to starting pressure (150 psi).

The reactor agitation was set to about 430 RPM. It was heated to 65° C. Hydrogen gas was cut off from the reactor. As pressure dropped, the reactor was re-pressurized several times with H₂. The temperature and pressure of H₂ was increased gradually to 100° C. and 300 psig. Once pressure stopped changing (i.e. hydrogen was no longer being consumed), the reaction was deemed complete.

After a period of time, the reactor was cooled and the pressure was vented.

A sample was analyzed by HPLC. The analysis results are shown in Table 24.

TABLE 24
Analytical results of Run 1.

| | 2-amino-N,3- | 2-nitro-N,3- |
|---|---|---|
| | dimethylbenzamide | dimethylbenzamide |
| % area | 55.9% | 8.2% |

The reaction mass was removed from the reactor. A second run of the reaction was then performed with the same catalyst. The reaction ran at 100° C. and 225 psig of H₂. The HPLC results are below in Table 25.

TABLE 25
Analytical results of Run 2.

| | 2-amino-N,3- | 2-nitro-N,3- |
|---|---|---|
| | dimethylbenzamide | dimethylbenzamide |
| % area | 84.69% | 5.93% |

The reaction mass was removed from the reactor. A third run of the reaction was then performed with the same catalyst. The reaction ran at 100° C. and 225 psig of H₂. The HPLC results are below in Table 26.

TABLE 26
Analytical results of Run 3.

| | 2-amino-N,3- | 2-nitro-N,3- |
|---|---|---|
| | dimethylbenzamide | dimethylbenzamide |
| % area | 85.0% | N/A |

After the third run, a weight % analysis was performed on the product mixture. The third reaction solution yield was about 95%, based on wt % assay.

TABLE 27
Weight % Analysis of Run 3.

| | 2-amino-N,3- | sodium 3-methyl- |
|---|---|---|
| | dimethylbenzamide | 2-aminobenzoate |
| Appearance | (wt %) | (wt %) |
| Brown-Black semi- | 12.53 | 0.61 |
| transparent solution | | |

The reaction selectivity for 2-amino-N,3-dimethylbenzamide is about 95.4% for the last run.

The three reactions completed within 300-600 min.

### Example 13. Reduction of 2-nitro-N,3-dimethylbenzamide to 2-amino-N,3-dimethylbenzamide Using a Nickel Pellet Catalyst

TABLE 28
Reactants.

| | | Catalyst | |
|---|---|---|---|
| | Reactant | Nickel pellet catalyst | |
| | 2-nitro-N,3- | (3 mm by 3 mm | Solvent |
| | dimethylbenzamide | cylinders) | Methanol |
| Molecular | 194.18 | | 32 |
| weight (g/mol) | | | |
| Mass (g) | 20 | 1 | 80 |

A 600 mL pressure reactor with overhead stirring and a spinning catalyst basket holder was charged with 2-nitro-N,3-dimethylbenzamide and methanol.

The spinning catalyst basket holder was charged with a pelletized nickel catalyst. The reactor was sealed and pressure-purged with N₂ three times to remove air. The reactor was pressure-tested using N₂. The reactor was then pressure-purged with hydrogen gas (H₂) three times. The reactor was pressurized with H₂ to starting pressure (150 psi).

The reactor agitation was set to about 430 RPM. It was heated to 65° C. Hydrogen gas was cut off from the reactor. As pressure dropped, the reactor was re-pressurized several times with H₂. Once pressure stopped changing and hydrogen was no longer being consumed, the reaction was deemed complete.

After a period of time, the reactor was cooled and the pressure was vented. The reaction mass was brown and transparent.

A sample was analyzed by HPLC. The analysis results are shown in Table 29.

TABLE 29
Analytical results.

| | 2-amino-N,3- | 2-nitro-N,3. |
|---|---|---|
| | dimethylbenzamide | dimethylbenzamide |
| % area | 85.0% | 9.8% |

This written description uses examples to illustrate the present disclosure, including the best mode, and also to enable any person skilled in the art to practice the disclosure, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of preparing a compound of Formula III, wherein 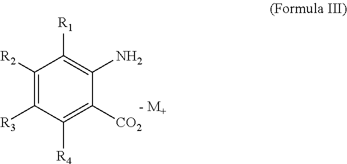
each of R₁-R₄ is independently selected from hydrogen and C₁-C₅ alkyl; and
M is a metal ion selected from sodium, potassium, calcium, and barium, the method comprising: reacting a mixture comprising
A) a compound of Formula II, wherein 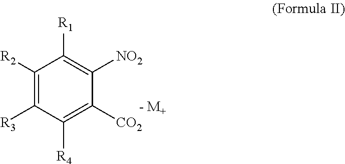
each of R₁-R₄ is independently selected from hydrogen and C₁-C₅ alkyl; and
M is a metal ion selected from sodium, potassium, calcium, and barium;
B) a reducing agent;
C) a catalyst; and
D) an aqueous solution.

2. The method of claim 1, wherein the reducing agent is hydrogen gas (H₂).

3. The method of claim 1, wherein the catalyst is in a form selected from a slurry, a pellet, a solid, a fine-grained solid, and combinations thereof.

4. The method of claim 1, wherein the catalyst is selected from nickel, Raney nickel, palladium, platinum, rhodium, gold, ruthenium, iridium, osmium, rhenium, silver, indium, germanium, beryllium, gallium, tellurium, bismuth, mercury, titanium, zirconium, hafnium, vanadium, niobium, tantalum, chromium, molybdenum, tungsten, manganese, technetium, iron, cobalt, copper, zinc, cadmium, and combinations thereof.

5. The method of claim 1, wherein the catalyst is selected from nickel, Raney nickel, palladium, platinum, Pd/C, and combinations thereof.

6. The method of claim 1, wherein the catalyst is dispersed on a support selected from metal oxides, zeolites, alumina, silicon carbide, carbons, and combinations thereof.

7. The method of claim 1, wherein the metal oxides are selected from Al₂O₃, SiO₂, TiO₂, and combinations thereof.

8. The method of claim 1, wherein the aqueous solution is selected from deionized water, tap water, and combinations thereof.

9. The method of claim 1, wherein the aqueous solution comprises a metal hydroxide.

10. The method of claim 1, wherein the aqueous solution is free of compounds selected from organic solvents, organic hydroxides, alkyl hydroxides, methanol, ethanol, isopropanol, and combinations thereof.

11. The method of claim 1, wherein the method step of reacting occurs at a temperature in the range of from about 80° C. to about 120° C.

12. The method of claim 1, wherein the method step of reacting occurs at a pressure in the range of from about 100 psi to about 400 psi.

13. The method of claim 1, wherein the compound of Formula III is

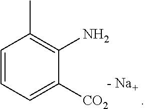

14. The method of claim 1, wherein the compound of Formula II is

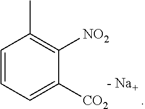

15. The method of claim 1, wherein the compound of Formula II is prepared according to a method comprising: dissolving a compound of Formula I, wherein 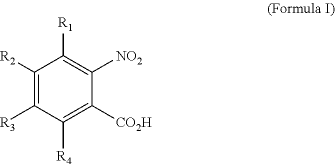
each of R₁-R₄ is independently selected from hydrogen and C₁-C₅ alkyl, in a mixture comprising
A) the compound of Formula I;
B) an aqueous solution; and
C) a metal hydroxide.

16. The method of claim 15, wherein the aqueous solution is selected from deionized water, tap water, and combinations thereof.

17. The method of claim 15, wherein the aqueous solution is free of compounds selected from organic solvents, organic hydroxides, alkyl hydroxides, methanol, ethanol, isopropanol, and combinations thereof.

18. The method of claim 15, wherein the metal hydroxide is sodium hydroxide.

19. The method of claim 15, wherein the compound of Formula II is

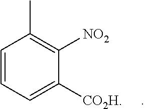

20. A method of preparing a compound of Formula V, wherein 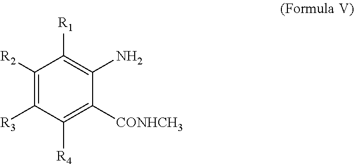
each of R₁-R₄ is independently selected from hydrogen and C₁-C₅ alkyl, the method comprising: reacting a mixture comprising
A) a compound of Formula IV, wherein 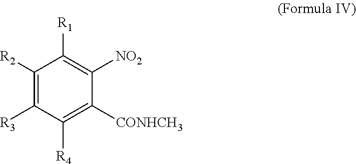
each of R₁-R₄ is independently selected from hydrogen and C₁-C₅ alkyl;
B) a reducing agent;
C) a catalyst; and
D) optionally an organic solvent.
